(19) United States
(12) Patent Application Publication
(10) Pub. No.: US 2021/0062007 A1
(43) Pub. Date: Mar. 4, 2021

(54) GARDENIA RED PIGMENT STABLY ADSORBED ON SURFACE OF SILICA, AND COSMETIC COMPOSITION CONTAINING SAME

(71) Applicant: Amorepacific Corporation, Seoul (KR)

(72) Inventors: Jae Won YOO, Yongin-si (KR); Mi Sook SHIN, Yongin-si (KR); Yong Jin KIM, Yongin-si (KR); Tae Ryong LEE, Yongin-si (KR)

(73) Assignee: Amorepacific Corporation, Seoul (KR)

(21) Appl. No.: 17/046,543

(22) PCT Filed: Apr. 12, 2019
(86) PCT No.: PCT/KR2019/004443

§ 371 Date: Oct. 9, 2020

(30) Foreign Application Priority Data
Apr. 13, 2018 (KR) 10-2018-0043522

(51) Int. Cl. C09C 3/08; A61Q 1/08; A61Q 1/06; A61Q 1/10; A61K 8/25

(52) U.S. Cl. CPC C09C 3/08; A61Q 1/08; A61K 2800/43; A61Q 1/10; A61K 8/25; A61Q 1/06

(57) ABSTRACT

One aspect of the present disclosure relates to a gardenia red pigment stably adsorbed on the surface of silica, and a cosmetic composition containing the same, and more specifically, to a gardenia red pigment stably adsorbed on the surface of silica, the silica surface having a specific reactive group introduced thereto, and a cosmetic composition containing the same. Compared to conventional gardenia red pigments, the gardenia red pigment stably adsorbed on the surface of silica, according to one aspect of the present disclosure, has improved thermal stability, does not aggregate at a low pH level, and may be applied to cosmetic compositions other than water-phase cosmetic compositions, and thus can have a further expanded range of use.

### TECHNICAL FIELD

One aspect of the present disclosure relates to a gardenia red pigment stably adsorbed on the surface of silica, and a cosmetic composition containing the same, and more specifically, to a gardenia red pigment stably adsorbed on the surface of silica, the silica surface having a specific reactive group introduced thereto, and a cosmetic composition containing the same.

### BACKGROUND ART

Natural pigments are defined as “materials used in addition, mixing, wetting or other methods for manufacturing, processing, and preserving foods” under the Food Sanitation Act. According to the US Food, Drugs and Cosmetic Act (FD & C Act), natural pigments are materials that can develop color, and are applied to foods, cosmetics, and pharmaceuticals in the form of coloring matters, pigments, and other materials. Recently, the field of application of these natural pigments has rapidly spread to textiles, daily necessities, building materials, interior goods, stationery, toys and the like, beyond foods, cosmetics, and pharmaceuticals, and accordingly, the definition of natural pigments is also changing.

Unlike synthetic and tar pigments, natural pigments are highly safe and thus highly reliable to consumers, and have many types of color tones and thus can be combined in various ways. In particular, synthetic pigments or synthetic dyes generate considerable pollution and wastewater during the manufacturing process, but natural pigments or natural dyes have a great advantage because they are non-toxic and thus very friendly to humans and environments.

However, in the case of natural pigments, the stability is poor compared to synthetic pigments. Thus, when the natural pigment extract itself is used or when it is used for various formulations, it is difficult to exert its function in terms of stability and thus it is not widely used. Therefore, there is a need for natural pigments with improved stability.

On the other hand, gardenia red pigment is one of the non-tar based natural pigments, and is obtained using the fruit extract of gardenia, and mainly used in beverages, processed livestock products, processed marine products, and noodle making. However, gardenia red pigment has a disadvantage in that it is slightly insufficient in thermal stability, especially it is aggregated at a low pH. In addition, since gardenia red pigment is water-soluble, there is a limitation that it can be used only for the water-phase among cosmetic compositions.

### PRIOR ART DOCUMENT

### Patent Document

Korean Patent Registration No. 10-1123394 Japanese Unexamined Patent Publication No. 2005-0336191

### DETAILED DESCRIPTION OF THE INVENTION

### Technical Problem

It is an object of one aspect of the present disclosure to provide a gardenia red pigment stably adsorbed on the surface of silica, the gardenia red pigment being stably adsorbed by introducing a specific reactive group on the silica surface, and thereby, having improved thermal stability compared to conventional existing gardenia red pigments, agglomeration phenomenon being not observed at low pH, and being appliable even to cosmetic compositions other than water-phase cosmetic compositions.

### Technical Solution

In order to achieve the above object, one aspect of the present disclosure provides a silica-adsorbed gardenia red pigment in which gardenia red pigment is adsorbed on the surface of silica by coupling with any one reactive group selected from the group consisting of an amine group, a carboxyl group, an ester group, a thiol group, a hydroxy group and hydrogen introduced on the surface of the silica. In the same context, one aspect of the present disclosure provides a gardenia red pigment-adsorbed silica in which gardenia red pigment is coupled with any one reactive group selected from the group consisting of an amine group, a carboxyl group, an ester group, a thiol group, a hydroxy group and hydrogen introduced on the surface of the silica.

Another aspect of the present disclosure provides a cosmetic composition containing the silica-adsorbed gardenia red pigment or the gardenia red pigment-adsorbed silica.

Yet another aspect of the present disclosure provides the use of the silica-adsorbed gardenia red pigment or the gardenia red pigment-adsorbed silica, more precisely the use as a color developing agent or a coloring agent, in the manufacture of basic cosmetic compositions, base makeup cosmetic compositions, tone cosmetic compositions, or cosmetic products.

### Advantageous Effects

Compared to conventional gardenia red pigments, the gardenia red pigment stably adsorbed on the surface of silica according to one aspect of the present disclosure has improved thermal stability, does not aggregate at a low pH level, and may be applied even to cosmetic compositions other than water-phase cosmetic compositions, and thus can have a further expanded range of use.

### BRIEF DESCRIPTION OF THE DRAWINGS

FIG. 1 shows the adsorption pattern of gardenia red pigment according to the presence or absence of the introduction of the reactive group on the surface of silica according to Test Example 1 of one aspect of the present disclosure.

### DETAILED DESCRIPTION OF THE EMBODIMENTS

Hereinafter, a gardenia red pigment stably adsorbed on the silica surface and a cosmetic composition containing the same according to one aspect of the present disclosure will be described in detail.

One aspect of the present disclosure provides a silica-adsorbed gardenia red pigment in which gardenia red pigment is adsorbed on the surface of silica by coupling with any one reactive group selected from the group consisting of an amine group, a carboxyl group, an ester group, a thiol group, a hydroxy group and hydrogen introduced on the surface of the silica.

In the same context, one aspect of the present disclosure provides a gardenia red pigment-adsorbed silica in which gardenia red pigment is coupled with any one reactive group selected from the group consisting of an amine group, a carboxyl group, an ester group, a thiol group, a hydroxy group and hydrogen introduced on the surface of the silica.

The silica may be replaced with inorganic particles such as titania and alumina.

In an exemplary embodiment of one aspect of the present disclosure, the silica may react with a silane compound represented by the following Chemical Formula 1 to introduce a reactive group.

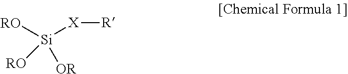

wherein, R represents a saturated or unsaturated, and linear or branched hydrocarbon having 1 to 23 carbon atoms, X represents a hydrocarbon having 1 to 12 carbon atoms, R′ represents —NR₁R₂, —COOR₁, —SH, —OH or —H, R₁ and R₂ represent each independently hydrogen, a hydrocarbon having 1 to 23 carbon atoms, or an alkylamine.

In an exemplary embodiment of one aspect of the present disclosure, the silica reacts with the silane compound represented by Chemical Formula 1 to introduce a reactive group (R′), and a gardenia red pigment is coupled to the reactive group (R′) via an ionic bond. In this case, preferably, the reactive is an amine group.

The silica-adsorbed gardenia red pigment of one aspect of the present disclosure can generally be manufactured by a method in which the reactive group-introduced silica is added to an aqueous solution in which the gardenia red pigment is dissolved, and the mixture is stirred and adsorbed, then filtered and washed with water to remove non-adsorbed gardenia red pigments, but the manufacturing method is not limited thereto.

The weight ratio of the gardenia red pigment and the reactive group-introduced silica constituting the silica-adsorbed gardenia red pigment according to one aspect of the present disclosure may be 0.01 to 40:60 to 99.99, preferably 0.1 to 20:80 to 99.9, and most preferably 1 to 15:85 to 99.

In addition, one aspect of the present disclosure provides a cosmetic composition containing the silica-adsorbed gardenia red pigment.

The cosmetic composition of one aspect of the present disclosure preferably contains 0.1 to 20% by weight of the silica-adsorbed gardenia red pigment based on the total weight of the composition. When the content is less than 0.1% by weight, it is difficult to properly obtain the desired effect such as color development of gardenia red pigment, and when the content exceeds 20% by weight, it is not easy to implement other functions of the product other than color development.

In the cosmetic composition according to one aspect of the present disclosure, the silica-adsorbed gardenia red pigment of one aspect of the present disclosure is mixed with additives such as oil, wax, water, etc., and can be used for basic cosmetics such as skins, lotions, creams, cosmetic lotions, essences, or packs, base makeup such as face powder, compact, powder foundation, two-way cake, makeup base, liquid foundation, cream foundation, and color tone cosmetics such as eye shadow, blusher, lipstick, eyeliner, mascara, and hair mascara, depending on the formulation or purpose of use.

Hereinbelow, one aspect of the present disclosure will be described in detail with accompanying Examples and Test Examples. However, these Examples are given for illustrative purposes only, and the scope of the invention is not intended to be limited by these Examples. Further, modifications, substitutions and insertions, etc. conventionally known in the art may be included, and such modifications are within the scope of the present disclosure.

### <Example 1> Preparation of Gardenia Red Pigment Adsorbed on Amine Group-Introduced Silica

Silica and a silane compound represented by the following Chemical Formula 2 were reacted in water or an organic solvent (ethanol or hexane) under reflux for 12 hours to prepare an amine group-introduced silica, and then, the amine group-introduced silica was added to an aqueous solution in which about 10% of gardenia red pigment relative to the mass of silica was dissolved in water of 5 times the mass of silica, and the mixture was stirred for 30 minutes, adsorbed, filtered, and then washed sequentially with an organic solvent (ethanol or hexane) and water to remove non-adsorbed gardenia red pigments, thereby preparing a gardenia red pigment (in a weight ratio of [gardenia red pigment]:[amine group introduced silica]=5:95) adsorbed on the amine group-introduced silica. (see Soto-Cantu E et al., Langmuir, 28(13):5562-9, 2012).

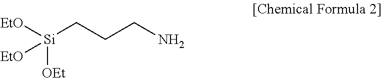 [SMILES: CCO[Si](CCCN)(OCC)OCC]

### <Example 2> Preparation of Gardenia Red Pigment Adsorbed on Amine Group-Introduced Silica

Silica and a silane compound represented by the following Chemical Formula 3 were reacted in water or an organic solvent (ethanol or hexane) under reflux for 12 hours to prepare an amine group-introduced silica, and then, the amine group-introduced silica was added to an aqueous solution in which about 10% of gardenia red pigment relative to the mass of silica was dissolved in water of 5 times the mass of silica, and the mixture was stirred for 30 minutes, adsorbed, filtered, and then washed sequentially with an organic solvent (ethanol or hexane) and water to remove non-adsorbed gardenia red pigments, thereby preparing a gardenia red pigment (in a weight ratio of [gardenia red pigment]:[amine group introduced silica]=5:95) adsorbed on the amine group-introduced silica.

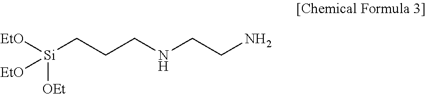 [SMILES: CCO[Si](CCCNCCN)(OCC)OCC]

### <Example 3> Preparation of Gardenia Red Pigment Adsorbed on Carboxyl Group-Introduced Silica

Silica and a silane compound represented by the following Chemical Formula 4 were reacted in water or an organic solvent (ethanol or hexane) under reflux for 12 hours to prepare a carboxyl group-introduced silica, and then, the carboxyl group-introduced silica was added to an aqueous solution in which about 10% of gardenia red pigment relative to the mass of silica was dissolved in water of 5 times the mass of silica, and the mixture was stirred for 30 minutes, adsorbed, filtered, and then washed sequentially with an organic solvent (ethanol or hexane) and water to remove non-adsorbed gardenia red pigments, thereby preparing gardenia red pigment adsorbed on the carboxyl group-introduced silica.

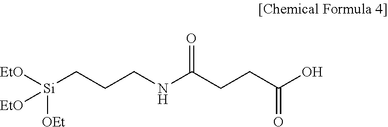 [SMILES: CCO[Si](CCCNC(=O)CCC(=O)O)(OCC)OCC]

### <Comparative Example 1> Preparation of Gardenia Red Pigment Adsorbed on General Silica

The gardenia red pigment adsorbed on general silica was prepared in the same manner as in Example 1, except that general silica to which a reactive group was not introduced was used.

### <Comparative Example 2> Preparation of Gardenia Red Pigment Adsorbed on Alkyl Group-Introduced Silica

Silica and a silane compound represented by the following Chemical Formula 5 were reacted in water or an organic solvent (ethanol or hexane) under reflux for 12 hours to prepare an alkyl group-introduced silica, and then, the alkyl group-introduced silica was added to an aqueous solution in which about 10% of gardenia red pigment relative to the mass of silica was dissolved in water of 5 times the mass of silica, and the mixture was stirred for 30 minutes, adsorbed, filtered, and then washed sequentially with an organic solvent (ethanol or hexane) and water to remove non-adsorbed gardenia red pigments, thereby preparing a gardenia red pigment adsorbed on the alkyl group-introduced silica.

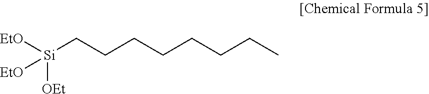 [SMILES: CCCCCCCC[Si](OCC)(OCC)OCC]

### <Test Example 1> Comparative Test of Adsorption Pattern of Gardenia Red Pigment

In order to compare the adsorption pattern (adsorption degree) of a gardenia red pigment adsorbed on the amine group-introduced silica of Example 1, and a gardenia red pigment adsorbed on the general silica of Comparative Example 1, the gardenia red pigment was dissolved in water at a concentration of about 1 wt. %, and then the same amount of silica of Example 1 and Comparative Example 1 was added, and left at room temperature. After about 30 minutes, the colors of the aqueous solution containing the gardenia red pigment and the amine group-introduced silica (Example 1) and the aqueous solution containing the gardenia red pigment and general silica (Comparative Example 1) were compared.

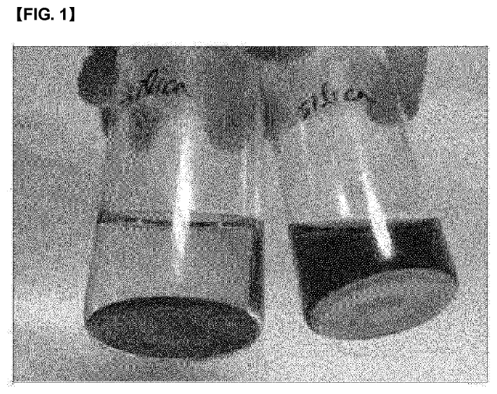

As a result, as shown in FIG. 1 below, it was confirmed that in the case of general silica, the adsorption was not performed well, so that the color of the aqueous solution containing gardenia red pigment and general silica was dark, and the color of the silica settled on the bottom was very pale, whereas in the case of a silica to which an amine group was introduced as a reactive group, the adsorption was performed stably and well, so that the color of the aqueous solution containing the gardenia red pigment and the amine group-introduced silica was lightened, and the color of the silica settled on the bottom was darkened.

The aqueous solution containing gardenia red pigment and general silica and the aqueous solution containing gardenia red pigment and amine group-introduced silica were filtered to remove the solution, and then the remaining silica was washed with water, and the results of comparing the color of silica are shown in Table 1 below.

TABLE 1

| Category | Silica color |
|---|---|
| Comparative Example 1 | X |
| Comparative Example 2 | X |
| Example 1 | ◯ |
| Example 2 | ◯ |
| Example 3 | Δ |

(◯: dark color, Δ: light color, X: almost no color)

From the results of Table 1, it was confirmed that silica to which an amine group was introduced as a reactive group was adsorbed clearly and stably with a gardenia red pigment as compared with a general silica to which a reactive group was not introduced and silica to which an alkyl group was introduced. In addition, it was confirmed that silica to which a reactive group other than an amine group was introduced also had a slight adsorption effect with the gardenia red pigment.

### <Test Example 2> Comparative Test of Thermal Stability

In order to confirm the thermal stability of the gardenia red pigment adsorbed on the amine group-introduced silica of Example 1, while the aqueous solution in which the silica-adsorbed gardenia red pigment of Example 1 was dispersed was stored at a temperature of about 40° C., the change in color over time was observed. In the control group, an aqueous solution in which gardenia red pigment not adsorbed on silica was dissolved was used. The results are shown in Table 2 below.

TABLE 2

| Color change | 1 week | 2 weeks | 3 weeks | 4 weeks |
|---|---|---|---|---|
| Gardenia red pigment | Δ | X | X | X |
| Example 1 | ◯ | ◯ | Δ | Δ |
| Example 2 | ◯ | ◯ | Δ | Δ |

(◯: no color change, Δ: slight color change but no big difference, X: clear color change)

As a result, as shown in Table 2, it was confirmed that the thermal stability of the gardenia red pigment adsorbed on the silica to which an amine group was introduced as a reactive group was significantly improved as compared with the non-adsorbed gardenia red pigment.

### <Test Example 3> Comparative Test of pH Stability

In order to confirm the pH stability of the gardenia red pigment adsorbed on the amine group-introduced silica of Example 1, the pH of the aqueous solution in which the silica-adsorbed gardenia red pigment of Example 1 was dispersed was adjusted, and after 2 weeks, aggregation or changes in properties were observed depending on the pH. In the control group, an aqueous solution in which gardenia red pigment not adsorbed on silica was dissolved was used. The results are shown in Table 3 below.

TABLE 3

| pH | 1 | 1.5 | 2 | 2.5 |
|---|---|---|---|---|
| gardenia red pigment | X | X | X | Δ |
| Example 1 | X | Δ | ◯ | ◯ |
| Example 2 | X | ◯ | ◯ | ◯ |

(◯: no change, Δ: aggregation or change in properties was observed, X: rapid aggregation or change in properties was observed)

As a result, as shown in Table 3, it was confirmed that the gardenia red pigment begins to show aggregation or change in properties below pH 2.5 and has low pH stability, whereas a gardenia red pigment adsorbed on the silica to which an amine group was introduced as a reactive group remains stable without change in properties at a lower pH as compared with gardenia red pigment.

Although specific parts of one aspect of the present disclosure have been described in detail, it will be apparent to those skilled in the art that these specific techniques are merely a preferred embodiment and that the scope of one aspect of the present disclosure is not limited thereto. Therefore, the substantial scope of one aspect of the present disclosure will be defined by the accompanying claims and their equivalents.

1. A silica-adsorbed gardenia red pigment, wherein the silica is surface-modified to contain a reactive group selected from the group consisting of an amine group, a carboxyl group, an ester group, a thiol group, a hydroxy group and hydrogen and wherein the gardenia red pigment is coupled to the reactive group.

2. The silica-adsorbed gardenia red pigment according to claim 1, wherein the surface modification of silica comprises reacting the silica with a silane compound of the following Chemical Formula 1: 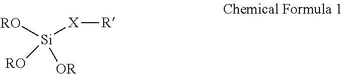
wherein, R represents a saturated or unsaturated, and linear or branched hydrocarbon having 1 to 23 carbon atoms, X represents a hydrocarbon having 1 to 12 carbon atoms, R′ represents —NR₁R₂, —COOR₁, —SH, —OH or —H, R₁ and R₂ represent each independently hydrogen, a hydrocarbon having 1 to 23 carbon atoms, or an alkylamine.

3. The silica-adsorbed gardenia red pigment according to claim 1, wherein the gardenia red pigment and the reactive group are coupled via an ionic bond.

4. The silica-adsorbed gardenia red pigment according to claim 1, wherein the reactive group is an amine group.

5. A cosmetic composition comprising the silica-adsorbed gardenia red pigment according to claim 1.

6. (canceled)

7. A cosmetic composition comprising the silica-adsorbed gardenia red pigment according to claim 2.

8. A cosmetic composition comprising the silica-adsorbed gardenia red pigment according to claim 3.

9. A cosmetic composition comprising the silica-adsorbed gardenia red pigment according to claim 4.

10. The cosmetic composition according to claim 5, which is a skincare product, a make-up product, or a hair-coloring product.

11. The cosmetic composition according to claim 7, which is a skincare product, a make-up product, or a hair-coloring product.

12. The cosmetic composition according to claim 8, which is a skincare product, a make-up product, or a hair-coloring product.

13. The cosmetic composition according to claim 9, which is a skincare product, a make-up product, or a hair-coloring product.
